(19) United States
(12) Patent Application Publication
(10) Pub. No.: US 2026/0061159 A1
(43) Pub. Date: Mar. 5, 2026

(54) EMBEDDED PULL-RING ASSEMBLY BRAIDING PROCESS

(71) Applicant: Boston Scientific Scimed, Inc., Maple Grove, MN (US)

(72) Inventors: Zhifeng Zhou, Misssissauga (CA); Erik Mroczkowski, Brampton (CA); Miles Montgomery, Holland Landing (CA)

(21) Appl. No.: 19/310,231

(22) Filed: Aug. 26, 2025

Related U.S. Application Data
(60) Provisional application No. 63/687,701, filed on Aug. 27, 2024.

(51) Int. Cl. A61M 25/00

(52) U.S. Cl. CPC A61M 25/0012; A61M 2207/10

(57) ABSTRACT

A system for applying a braid to a tubular member includes an infeed clamp, an infeed guide, and a nose cone. The infeed clamp is configured to prevent rotation of a workpiece, a first steering member, and a second steering member. The infeed guide is configured to maintain the first steering member and the second steering member in a desired axial location along the workpiece. The nose cone includes a proximal plate, a distal plate, and a radially expanding inner guiding member. The proximal plate and the distal plate form a recess when joined together, and the radially expanding inner guiding member is located in the recess.

### CROSS REFERENCE TO RELATED APPLICATIONS

The present application claims priority to U.S. Provisional Patent Application No. 63/687,701, filed Aug. 27, 2024, the entire disclosure of which is incorporated herein.

### TECHNICAL FIELD

The present disclosure relates to systems and methods for applying a braid to a tubular member. More specifically, the present disclosure relates to systems and methods for applying a braid over a tubular member having a pull ring and one or more steering member affixed to the pull ring.

### BACKGROUND

Various medical fields use different types of catheters to achieve access to a physiological site in medical procedures. For instance, electrophysiological procedures involve guiding catheters into the heart and tracking the location of the catheters with respect to the heart. Catheter ablation is minimally invasive electrophysiological procedure to treat a variety of heart conditions such as supraventricular and ventricular arrhythmia. Example catheters used in catheter ablation can include mapping catheters, ablation catheters, guiding sheaths or introducer sheaths, dilators, and other medical tools, which can be referred to as catheters in this disclosure.

To position a catheter within the body at a target site, some type of navigation mechanism is used, such as a steering feature incorporated into the catheter. Clinicians manually manipulate or operate the catheter using the steering feature. To facilitate the advancement of catheters through a patient's vasculature, the concurrent application of torque at a proximal portion of the catheter and the ability to deflect the distal portion of the catheter in a selected direction permits the clinician to adjust the direction of advancement of the distal portion of the catheter and to selectively position the distal portion of the catheter during an electrophysiological procedure. In an example, the distal portion includes a pull ring assembly having a pull ring that is deflected with a pull wire or other tension member attached or anchored at the distal end of the catheter and extending proximally to an actuator in a control handle that controls the application of tension on the pull wire. Often, a catheter is configured to deflect in more than one direction and includes a pair of pull wires installed on the pull ring on opposite side from each other.

### SUMMARY

Example 1 is a system for applying a braid to a tubular member. The system includes an infeed clamp, an infeed guide, and a nose cone. The infeed clamp is configured to prevent rotation of a workpiece and one or more steering member. The infeed guide is configured to maintain the one or more steering member in a desired axial location along the workpiece. The nose cone includes a radially expanding inner guiding member.

Example 2 is the system of Example 1, wherein the infeed clamp includes one or more steering member retainer and a workpiece retainer.

Example 3 is the system of any of Examples 1 or 2, wherein the infeed clamp includes at least one tensioner to apply tension to the one or more steering member.

Example 4 is the system of any of Examples 1-3, wherein the infeed clamp includes a cover, the cover being biased to an open position allowing for loading of the one or more steering member into the one or more steering member retainer, and the workpiece into the workpiece retainer.

Example 5 is the system of any of Examples 2-4, wherein the cover includes a closed position for securing the one or more steering member in the one or more steering member retainer, and the workpiece in the workpiece retainer.

Example 6 is the system of Example 5, wherein the infeed clamp includes a closure configured to secure the cover in the closed position.

Example 7 is the system of Example 6, wherein the infeed guide includes a stationary releasing element configured to initiate release of the closure, allowing the cover to assume the open position.

Example 8 is the system of Example 7, wherein the stationary releasing element is spaced from a side of the infeed guide and includes a ramped surface configured for contacting the closure.

Example 9 is the system of any of Examples 1-8, wherein the infeed guide includes an infeed surface and an outfeed surface, and a funnel shaped opening having one or more channel configured to receive the one or more steering member, the funnel shaped opening tapering from the infeed surface to the outfeed surface.

Example 10 is the system of any of Examples 1-9, wherein the expanding inner guiding member includes an upper half and a lower half, the upper half and the lower half being semicircular.

Example 11 is the system of any of Examples 1-10, wherein the expanding inner guiding member includes an outer circumferential groove, and a garter spring located in the outer circumferential groove.

Example 12 is the system of any of Examples 10 or 11, wherein the expanding inner guiding member includes at least one cylindrical recess, and at least one cylindrical guide located in the at least one cylindrical recess to align the upper half with the lower half.

Example 13 is the system of any of Examples 1-12, wherein the expanding inner guiding member includes a flared infeed opening and a flared outfeed opening, the flared infeed opening and the flared outfeed opening being joined by a constant diameter lumen.

Example 14 is the system of Example 13, wherein the flared infeed opening, the flared outfeed opening, and the constant diameter lumen include one or more channel configured to receive the one or more steering member.

Example 15 is the system of any of Examples 1-14, wherein the nose cone includes a proximal plate and a distal plate, the proximal plate and the distal plate forming a recess when joined together, and the expanding inner guiding member being located in the recess.

Example 16 is a system for applying a braid to a tubular member. The system includes an infeed clamp, an infeed guide, and a nose cone. The infeed clamp is configured to prevent rotation of a workpiece, a first steering member, and a second steering member. The infeed guide is configured to maintain the first steering member and the second steering member in a desired axial location along the workpiece. The nose cone includes a proximal plate, a distal plate, and a radially expanding inner guiding member. The proximal plate and the distal plate form a recess when joined together, and the radially expanding inner guiding member is located in the recess.

Example 17 is the system of Example 16, wherein the infeed clamp includes a first steering member retainer, a second steering member retainer, and a workpiece retainer.

Example 18 is the system of Example 16, wherein the infeed clamp includes at least one tensioner to apply tension to the first steering member or the second steering member.

Example 19 is the system of Example 17, wherein the infeed clamp includes a cover, the cover being biased to an open position allowing for loading of the first steering member into the first steering member retainer, the second steering member into the second steering member retainer, and the workpiece into the workpiece retainer.

Example 20 is the system of Example 19, wherein the cover includes a closed position for securing the first steering member in the first steering member retainer, the second steering member in the second steering member retainer, and the workpiece in the workpiece retainer.

Example 21 is the system of Example 20, wherein the infeed clamp includes a closure configured to secure the cover in the closed position.

Example 22 is the system of Example 21, wherein the infeed guide includes a stationary releasing element configured to initiate release of the closure, allowing the cover to assume the open position.

Example 23 is the system of Example 22, wherein the stationary releasing element is spaced from a side of the infeed guide and includes a ramped surface configured for contacting the closure.

Example 24 is the system of Example 16, wherein the infeed guide includes an infeed surface and an outfeed surface, and a funnel shaped opening having a first channel configured to receive the first steering member and a second channel configured to receive the second steering member, the funnel shaped opening tapering from the infeed surface to the outfeed surface.

Example 25 is the system of Example 16, wherein the expanding inner guiding member includes an upper half and a lower half, the upper half and the lower half being semicircular.

Example 26 is the system of Example 25, wherein the expanding inner guiding member includes an outer circumferential groove, and a garter spring located in the outer circumferential groove.

Example 27 is the system of Example 25, wherein the expanding inner guiding member includes at least one cylindrical recess, and at least one cylindrical guide located in the at least one cylindrical recess to align the upper half with the lower half.

Example 28 is the system of Example 16, wherein the expanding inner guiding member includes a flared infeed opening and a flared outfeed opening, the flared infeed opening and the flared outfeed opening being joined by a constant diameter lumen.

Example 29 is the system of Example 28, wherein the flared infeed opening, the flared outfeed opening, and the constant diameter lumen include a first channel configured to receive the first steering member and a second channel configured to receive the second steering member.

Example 30 is an infeed clamp for use with a system for applying a braid to a tubular member. The infeed clamp includes a first steering member retainer, a second steering member retainer, and a workpiece retainer. The infeed clamp includes a cover having an open position and a closed position. The cover is biased to the open position for allowing loading of the first steering member into the first steering member retainer, the second steering member into the second steering member retainer, and the workpiece into the workpiece retainer. The closed position is configured for securing the first steering member in the first steering member retainer, the second steering member in the second steering member retainer, and the workpiece in the workpiece retainer.

Example 31 is the clamp of Example 30, further comprising at least one tensioner to apply tension to the first steering member or the second steering member.

Example 32 is the claim of Example 30, further comprising a closure configured to secure the cover in the closed position.

Example 33 is a nose cone for use with a system for applying a braid to a tubular member. The nose cone includes a proximal plate configured to attach to a portion of the system for applying a braid to a tubular member. The nose cone includes a distal plate configured to join to the proximal plate. The nose cone includes a radially expanding inner guiding member. The proximal plate and the distal plate form a recess when joined together. The radially expanding inner guiding member is located in the recess.

Example 34 is the nose cone of Example 33, wherein the expanding inner guiding member includes an upper half movable from a lower half; an outer circumferential groove located in the upper half and the lower half; and a garter spring located in the outer circumferential groove configured to bias the upper half and the lower half together.

Example 35 is the nose cone of Example 33, wherein the expanding inner guiding member includes a flared infeed opening and a flared outfeed opening, the flared infeed opening and the flared outfeed opening being joined by a constant diameter lumen; the flared infeed opening, the flared outfeed opening, and the constant diameter lumen include a first channel configured to receive a first steering member and a second channel configured to receive a second steering member.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic overview of a system for applying a braid to a tubular member, in accordance with embodiments of the disclosure.

FIG. 2 is perspective view illustrating an infeed portion of the system of FIG. 1, in accordance with embodiments of the disclosure.

FIG. 3 is a perspective view illustrating an infeed clamp for use with the system of FIG. 1, in accordance with embodiments of the disclosure.

FIGS. 4A-4C are schematic illustrations of an alternate infeed clamp for use with the system of FIG. 1, in accordance with embodiments of the disclosure.

FIG. 5 is a perspective view illustrating an alternate infeed clamp for use with the system of FIG. 1, in accordance with embodiments of the disclosure.

FIG. 6 is an additional perspective view illustrating the alternate infeed clamp of FIG. 5, in accordance with embodiments of the disclosure.

FIG. 7 is a perspective view of an infeed guide for use with the system of FIG. 1, in accordance with embodiments of the disclosure.

FIG. 8 is a perspective view illustrating a nose cone for use with the system of FIG. 1, in accordance with embodiments of the disclosure.

FIG. 9 is a cross-sectional view of the inner guiding member of the nose cone of FIG. 6, in accordance with embodiments of the disclosure.

FIG. 10A is a perspective view of the inner guiding member of the nose cone expanding as it moves over a pull ring, in accordance with embodiments of the disclosure.

FIG. 10B is a perspective view of the inner guiding member of the nose cone after passing over the pull ring, in accordance with embodiments of the disclosure.

FIG. 11 is a perspective view of an alternative nose cone for use with the system of FIG. 1, in accordance with embodiments of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

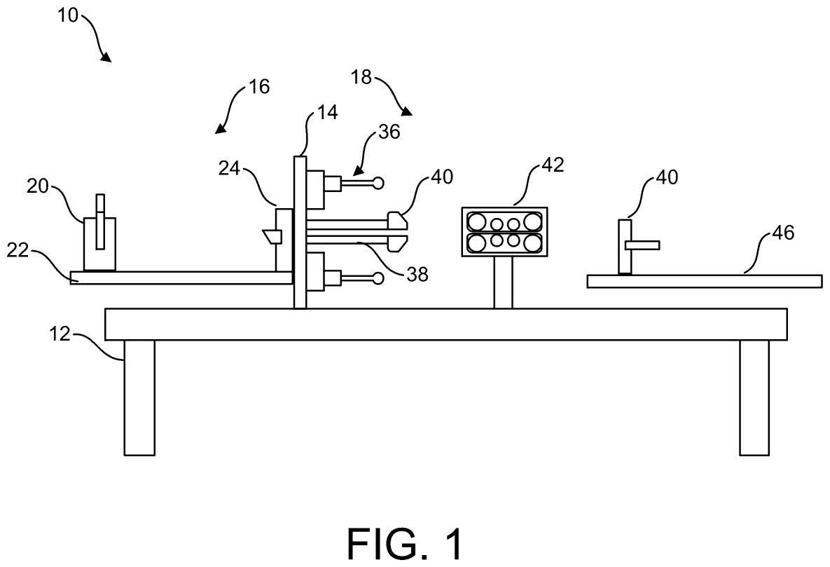

FIG. 1 is a schematic overview of a system 10 for applying a braid to a tubular member, in accordance with embodiments of the disclosure. The system includes a support 12 configured to securely support the components of the system 10. The support 12 can take the form of a table, a series of rails joined together, or other structure capable of supporting the components of the system 10. The system 10 includes a wall 14 that separates an infeed portion 16 from an outfeed portion 18. As a tubular member is braided, it moves from the infeed portion 16 towards the outfeed portion 18.

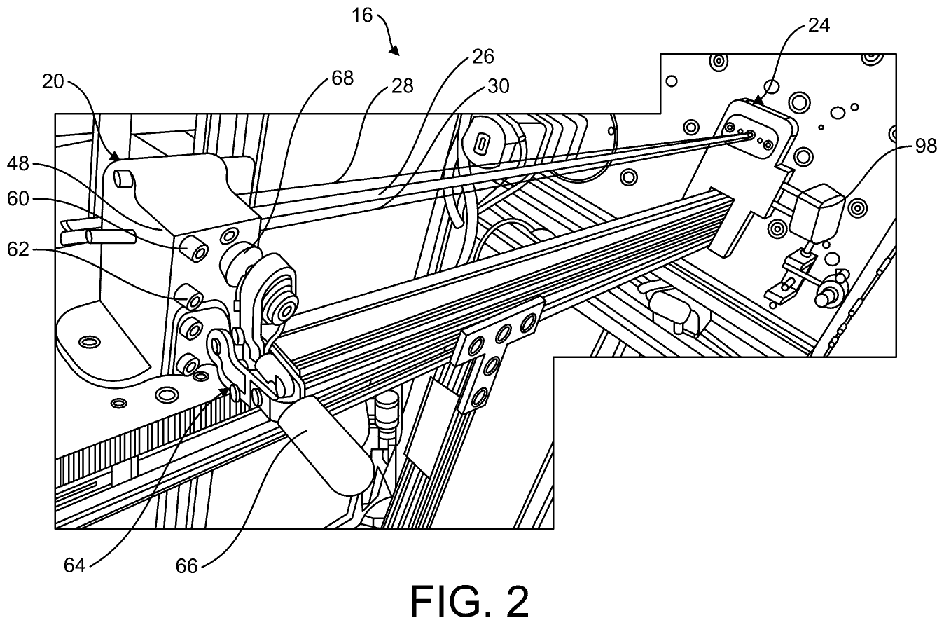

The infeed portion 16 includes an infeed clamp 20 mounted on an infeed rail 22 and an infeed guide 24 attached to the wall 14. The infeed clamp 20 is configured to move along the infeed rail 22 towards the infeed guide 24 during the braiding process. As illustrated in FIG. 2, the infeed clamp 20 is configured to prevent rotation of a workpiece 26, and one or more steering member during the braiding process. A workpiece 26 includes a tubular member on which a braid is to be applied as well as a support piece. For example, a workpiece 26 can include a stainless-steel mandrel, a tubular member, or a tubular member mounted on a stainless-steel mandrel. The infeed guide 24 is configured to maintain the one or more steering member in a desired axial location along the workpiece 26. The one or more steering member can include a single steering member or a plurality of steering members. For example, the one or more steering member can include a first steering member 28 and a second steering member 30.

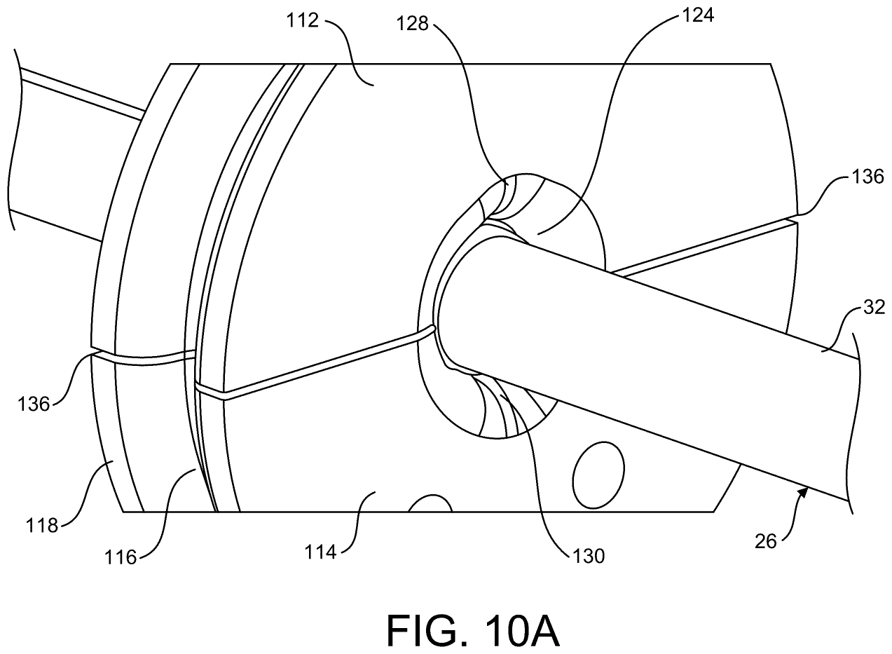

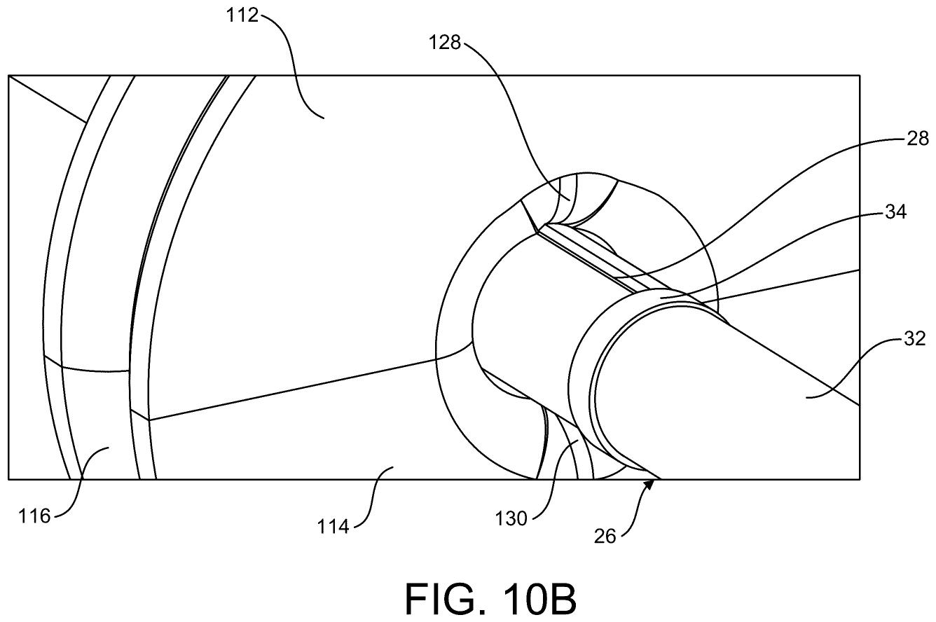

Before being placed in the system 10 for braiding, a tubular member 32 (See FIG. 10B), such as a sheath, catheter, dilator, lead, or other flexible, deflectable medical tool that can be steered to a desired target site within a patient, is joined with a pull ring 34 and one or more steering member. The pull ring 34 and one or more steering member may be attached to the tubular member 32. In some aspects, the pull ring 34 may be attached using adhesive, welding, crimping, or tacked using heat, such as hot air. The pull ring 34 is then fixed in relation to the tubular member 32 and does not translate longitudinally along the tubular member 32.

As illustrated, the pull ring 34 is joined with the first steering member 28 and the second steering member 30. The first steering member 28 and the second steering member 30 are attached to diametrically opposed locations on a face of the pull ring 34 and must remain axially in these locations along the length of the tubular member 32 in order to prevent kinks or bends as a braid is being wound over the first and second steering members 28, 30. The first and second steering members 28, 30 can include a steering wire or a sheath containing a steering wire. Once assembled, a tension applied to a proximal end of the first steering member 28 or the second steering member 30, for example by an actuator or handle, will result in deflection of the tubular member 32. Thus, allowing for steering of the tubular member 32 during a medical procedure.

The outfeed portion 18 of the system 10 includes a braiding machine 36 having a spindle 38 through which the workpiece 26 passes during application of the braid. The spindle 38 may include magnets to aid in alignment of one or more steering member along the tubular member 32. Spindle 38 is configured to translate axially toward the infeed portion 16 or toward the outfeed portion 18. As known in the art and not described herein, the braiding machine 36 includes one or more bobbins for holding a filament forming the braid, carriers to hold and guide the bobbins through the braiding process, and horn gears to drive the carriers in a specific pattern to create the desired braid. Located at a distal end of the spindle 38 is a nose cone 40 over which filaments slide during the braiding process. A belt driven conveyor 42 moves the workpiece 26 towards an outfeed clamp 44. The outfeed clamp 44 secures a distal end of the workpiece 26 and is translatable along an outfeed rail 46. As the conveyor 42 drives the workpiece 26, the outfeed clamp 44 holding the workpiece 26 moves along the outfeed rail 46 away from the wall 14.

FIG. 2 is perspective view illustrating the infeed portion 16 of the system 10 of FIG. 1, in accordance with embodiments of the disclosure. In FIG. 2, the infeed clamp 20 is shown holding the first steering member 28, the second steering member 30, and the workpiece 26. The first steering member 28, the second steering member 30, and the workpiece 26 are shown passing through the infeed guide 24 to the outfeed portion (not shown) of the system 10. The infeed clamp 20 is configured to apply a tension to the first steering member 28 and the second steering member 30 to prevent axial shifting of the first and second members 28, 30 during the braiding process.

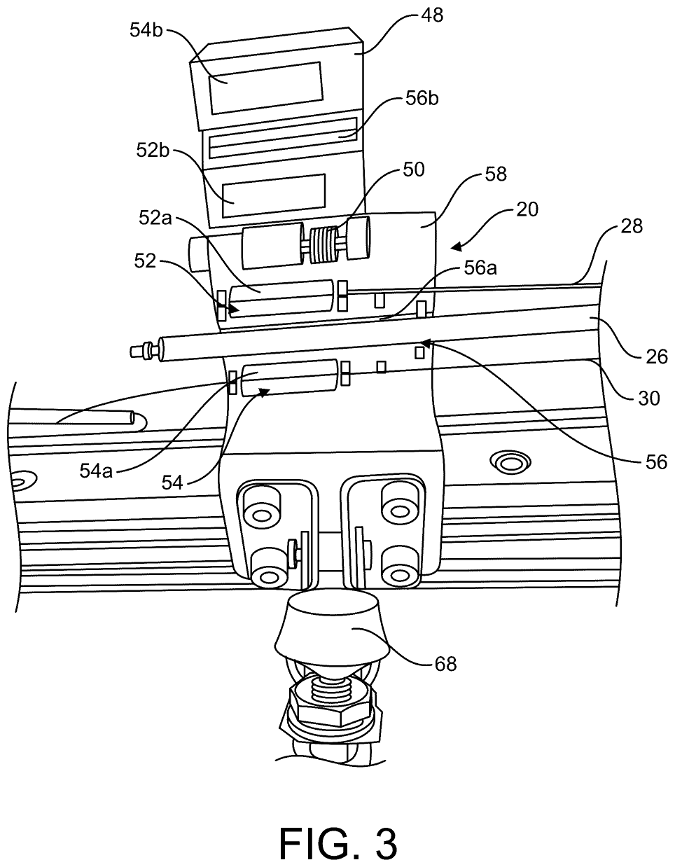

The infeed clamp 20 includes a cover 48 that is movable between an open position and a closed position. FIG. 3 illustrates the cover 48 in the open position. The cover 48 is biased by a spring 50 towards the open position. The open position allows for the loading and unloading of the first steering member 28 into a first steering member retainer 52, the second steering member 30 into a second steering member retainer 54, and the workpiece into a workpiece retainer 56. In the closed position, illustrated in FIG. 2, the cover 48 aids securing the first steering member 28 in the first steering member retainer 52, the second steering member 30 in the second steering member retainer 54, and the workpiece 26 in the workpiece retainer 56.

Each of the first steering member retainer 52, the second steering member retainer 54, and the workpiece retainer 56 include multiple pieces. As shown in FIG. 3, the workpiece retainer 56 includes a first piece 56a located on a base 58 of the infeed clamp 20 and a second piece 56b on an inner surface of the cover. Similarly, the first steering member retainer 52 includes a first piece 52a located on the base 58 of the infeed clamp 20 and a second piece 52b located on the inner surface of the cover 48. The first piece 52a and the second piece 52b are configured hold the first steering member 28 when the cover 48 is closed. The first piece 52a and/or the second piece 52b can be formed of a material with a high coefficient of friction, such as a rubber or polymer. A first steering member tensioner 60 is configured to apply a tension to the first steering member 28 located in the first steering member retainer 52. The first steering member tensioner 60 is configured to move the second piece 52b relative to the first piece 52a to apply a tension to the first steering member 28. In some embodiments, the tensioner 60 moves the second piece 52b closer or further from the first piece 52a. In other embodiments, the tensioner 60 slides the second piece 52b along a longitudinal axis of the first steering member 28. In some embodiments, the tensioner 60 both moves the second piece 52b closer or further from the first piece 52a and along a longitudinal axis of the first steering member 28.

Similarly, the second steering member retainer 54 includes a first piece 54a located on the base 58 of the infeed clamp 20 and a second piece 54b located on the inner surface of the cover 48. The first piece 54a and the second piece 54b are configured hold the second steering member 30 when the cover 48 is closed. The first piece 54a and/or the second piece 54b can be formed of a material with a high coefficient of friction, such as a rubber or polymer. A second steering member tensioner 62 is configured to apply a tension to the second steering member 30 located in the second steering member retainer 54. The second steering member tensioner 62 is configured to move the second piece 54b relative to the first piece 54a to apply a tension to the second steering member 30. In some embodiments, the second steering member tensioner 62 moves the second piece 54b closer or further from the first piece 54a. In other embodiments, the second steering member tensioner 62 slides the second piece 54b along a longitudinal axis of the second steering member 30. In some embodiments, the second steering member tensioner 62 both moves the second piece 54b closer or further from the first piece 54a and along a longitudinal axis of the second steering member 30.

The infeed clamp 20 includes a closure 64 configured to secure the cover 48 in the closed position. The closure 64 includes a pivoting arm 66 and a stopper 68. The pivoting arm 66 has a first position that pushes the stopper 68 against a surface of the cover 48 and a second position that pulls the stopper 68 away from the cover 48 such that the cover 48 opens as a result of the spring 50. After positioning the first steering member 28, the second steering member 30, and workpiece 26 in the clamp 20, a user closes the cover 48 and positions the pivoting arm 66 in the first position to place stopper 68 against the cover 48 to keep it closed and secure the first and second steering members 28, 30 and the workpiece 26 therein. In other embodiments, the closure 64 can include magnetic closing mechanisms, pneumatic closing mechanisms, or other mechanisms configured to ensure that the cover 48 remains in a closed position when desired.

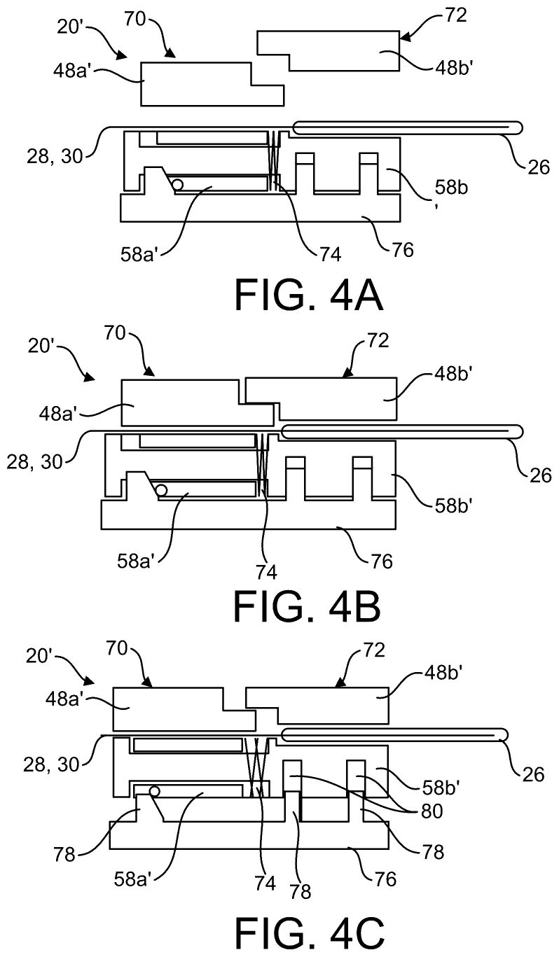

FIGS. 4A-4C are schematic illustrations of an alternate infeed clamp 20′ for use with the system 10 of FIG. 1, in accordance with embodiments of the disclosure. Infeed clamp 20′ includes a first portion 70 that is movable relative to a second portion 72. The first portion 70 includes a base 58a′ and a cover 48a′. The second portion 72 includes a base 58b′ and a cover 48b′. The second portion cover 48b′ is configured to interact with second portion base 58b′ to secure a workpiece 26 therein. The first portion cover 48a′ is configured to interact with the first portion base 58a′ to secure a first steering member 28 and a second steering member 30 therein. A spring 74 causes the first portion 70 to move relative to the second portion 72 to allow for tensioning of the first and second steering members 28, 30. A plate 76 is configured to hold the first portion 70 and the second portion 72 stationary during loading. The plate 76 includes protrusions 78 that fit within recesses 80 in the first portion base 58a′ or the second portion base 58b′ or block a portion of the first portion base 58a′ or the second portion base 58b′ to keep the spring 74 compressed. Once the first steering member 28, the second steering member 30, and the workpiece 26 are loaded into the infeed clamp 20′, the plate 76 is removed allowing relative movement between the first portion 70 and the second portion 72 and thus tensioning of the first and second steering members 28, 30. In other embodiments, the plate 76 may not include protrusions 78, but may include one or more portions that surround the first portion base 58a′ and the second portion base 58b′ to keep the first portion base 58a′ adjacent to or in contact with the second portion base 58b′. Additionally, the plate 76 may include adhesives, materials having a high coefficient of friction, or magnets to maintain the first portion base 58a′ in a stationary position relative to the second portion base 58b′.

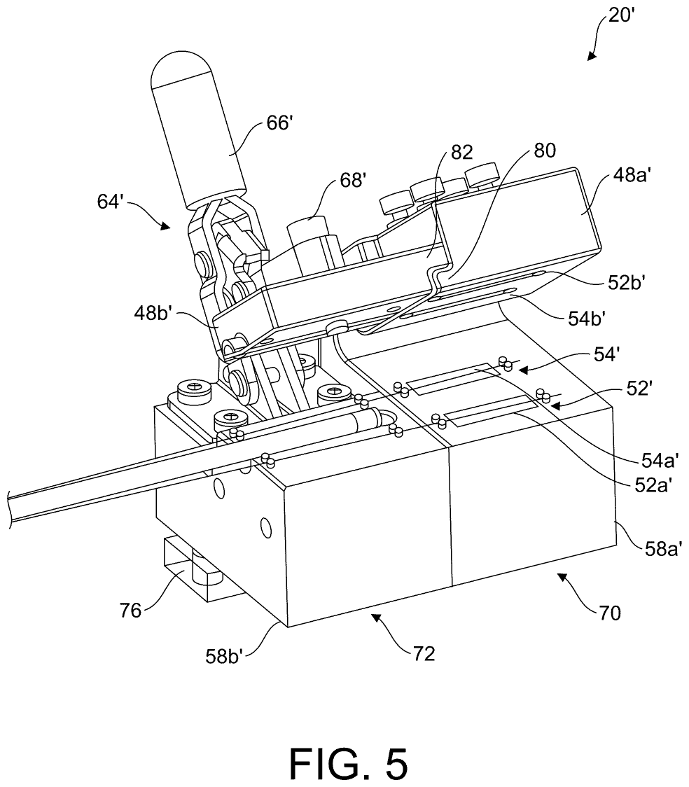

FIG. 5 is a perspective view of the alternate infeed clamp 20′, in accordance with embodiments of the disclosure. FIG. 5 illustrates the first portion cover 48a′ and the second portion cover 48b′ in an open position. The plate 76 is secured to the bottom of the first portion 70 and the second portion 72. In this arrangement, the first portion 70 is adjacent the second portion 72 and the first portion 70 is fixed relative to the second portion 72 to allow for loading of the first and second steering member 28, 30 and the workpiece 26. The infeed clamp 20′ includes a first steering member retainer 52′, a second steering retainer 54′, and a workpiece retainer 56′. The first and second steering member retainers 52′, 54′ are located within the first portion 70. The workpiece retainer 56′ is located within the second portion 72. Each of the first steering member retainer 52′, the second steering member retainer 54′, and the workpiece retainer 56′ include multiple pieces.

As shown in FIG. 5, the first steering member retainer 52′ includes a first piece 52a′ located on the first portion base 58a′ of the infeed clamp 20′ and a second piece 52b′ located on an inner surface of the first portion cover 48a′. The first piece 52a′ and the second piece 52b′ are configured hold the first steering member 28 when the first portion cover 48a′ is closed. The first piece 52a′ and/or the second piece 52b′ can be formed of a material with a high coefficient of friction, such as a rubber or polymer. The second steering member retainer 54′ includes a first piece 54a′ located on the first portion base 58a′ of the infeed clamp 20′ and a second piece 54b′ located on the inner surface of the first portion cover 48a′. The first piece 54a′ and the second piece 54b′ are configured hold the second steering member 30 when the first portion cover 48a′ is closed. The first piece 54a′ and/or the second piece 54b′ can be formed of a material with a high coefficient of friction, such as a rubber or polymer.

The workpiece retainer 56′ includes a first piece 56a′ located on the second portion base 58b′ of the infeed clamp 20 and a second piece 56b′ on an inner surface of the second portion cover 48b′. The first piece 56a′ and/or the second piece 56b′ can be formed of a material with a high coefficient of friction, such as a rubber or polymer.

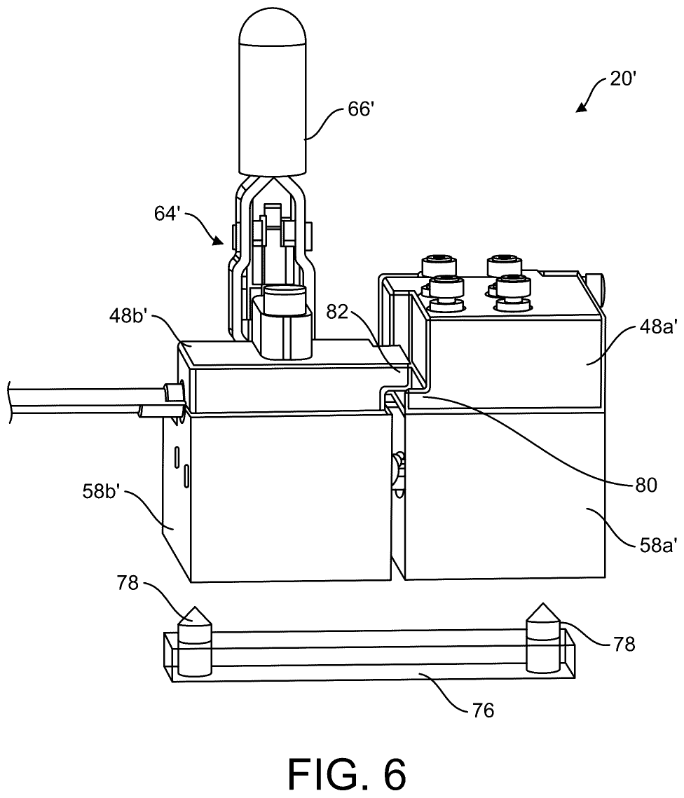

FIG. 6 is an additional perspective view illustrating the first portion cover 48a′ and the second portion cover 48b′ of the infeed clamp 20′ in a closed position. The infeed clamp 20′ includes a closure 64′ configured to secure the first portion cover 48a′ and the second portion cover 48b′ in the closed position. The closure 64′ includes a pivoting arm 66′ and a connector 68′ that is attached to an outer surface of the second portion cover 48b′. The first portion cover 48a′ and the second portion cover 48b′ are movable between and open position and a closed position together although they are not connected. The first portion cover 48a′ includes a lower shoulder 80 that is overlapped by an upper shoulder 82 of the second portion cover 48b′. As such, when the pivoting arm 66′ is moved to a first position causing movement of the second portion cover 48′ towards a closed position, the first portion cover 48a′ is moved to a closed position by virtue of an interaction between the upper shoulder 82 on the lower shoulder 80. During opening of the infeed clamp 20′, movement of the pivoting arm 66′ to a second position opens the second portion cover 48b′ and a spring (not shown) biases the first portion cover 48a′ to the open position. In other embodiments, the closure 64′ can include magnetic closing mechanisms, pneumatic closing mechanisms, or other mechanisms configured to ensure that the first portion cover 48a′ and the second portion cover 48b′ remain in a closed position when desired.

As illustrated in FIG. 6, the plate 76 is removed from the bottom of the first portion 70 and the second portion 72. This allows the spring 74 to push the first portion 70 away from the second portion 72 to cause tensioning of the first and second steering members 28, 30. The lower shoulder 80 and the upper shoulder 82 have a length that is longer than a distance of separation between the first portion 70 and the second portion 72. This ensures that the upper shoulder 82 remains over the lower shoulder 80 such that the first portion cover 48a′ remains closed.

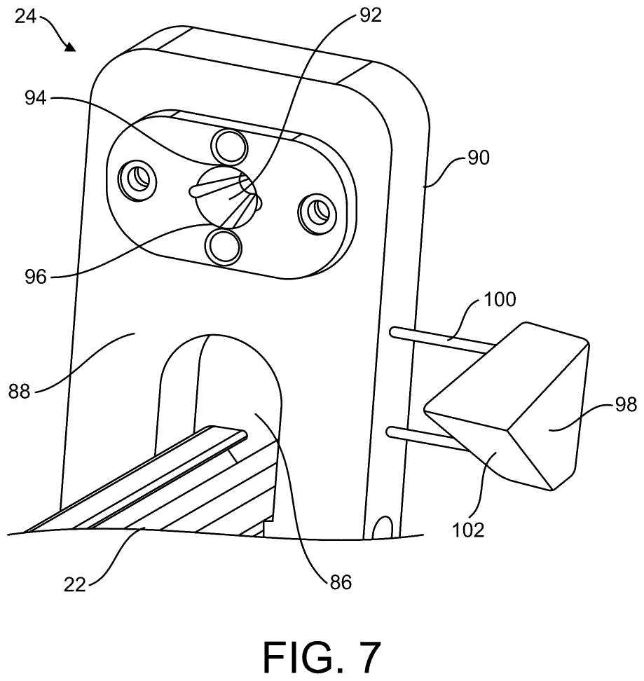

FIG. 7 is a perspective view of an infeed guide 24 for use with the system 10 of FIG. 1, in accordance with embodiments of the disclosure. The infeed guide 24 is configured to ensure that the first and second steering members 28, 30 remain in the same axial location along the length of the tubular member 32 during the braiding process. The infeed guide 24 includes a rectangular body 84 having a lower trough 86 configured to receive a portion of the infeed rail 22. The infeed guide 24 includes an infeed surface 88 facing the infeed portion 16 of the system 10 and an outfeed surface 90 facing the outfeed portion 18 of the system. In some embodiments, the outfeed surface 90 is attached to the wall 14. An upper portion of the body 84 includes a funnel shaped opening 92. The funnel shaped opening 92 includes a first channel 94 configured to receive the first steering member 28 and a second channel 96 configured to receive the second steering member 30. The funnel shaped opening 92 tapers in diameter from a larger diameter at the infeed surface 88 to a smaller diameter at the outfeed surface 90.

The infeed guide 24 includes a stationary releasing element 98 configured to initiate release of the closure 64, allowing the cover 48 to assume the open position. The stationary releasing element 98 is configured to cause the pivoting arm 66 to move from the first position to the second position. The stationary releasing element 98 is shown spaced from a side of the infeed guide 24 by a pair of supports 100. However, in some embodiments, the stationary releasing element 98 is adjacent the rectangular body 84. The stationary releasing element 98 includes a body having a ramped surface 102 configured for contacting the pivoting arm 66. The body can include a triangular or polygonal shape. During the braiding process, the infeed clamp 20 is pulled towards the infeed guide 24 by the conveyor 42. When the pivoting arm 66 is pulled into the ramped surface 102, the arm 66 is forced into the second position. This allows the cover 48 to be automatically opened by virtue of the spring 50 and release of the first and second steering members 28, 30 and the workpiece 26. The first and second steering members 28, 30 and the workpiece 26 are then pulled through the wall 14 to the outfeed portion 18 of the system 10. While the stationary releasing element 98 illustrated in FIG. 7 is configured to mechanically interact with the arm 66 to move the arm to the second position, other embodiments may utilize other mechanisms for initiating release of the closure 64. For example, in embodiments where the closure 64 includes magnetic, electromagnetic, or pneumatic devices, a location sensor may detect that the infeed clamp 20 has reached a desired location or position along the infeed rail 22 and automatically trigger release of the closure 64. In other embodiments, a proximity sensor associated with the stationary releasing element 98, such as a magnetic or light sensor, may detect that the infeed clamp 20 is in proximity to the stationary releasing element 98 and automatically trigger release of the closure 64.

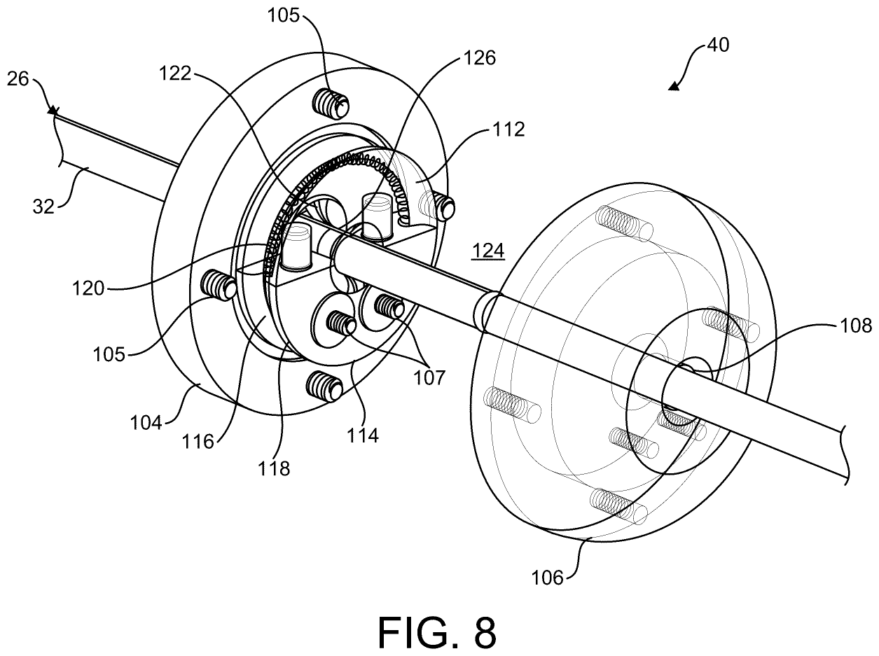

FIG. 8 is a perspective view illustrating a nose cone 40 for use with the system 10 of FIG. 1, in accordance with embodiments of the disclosure. The nose cone 40 includes a proximal plate 104 and a distal plate 106. The proximal plate 104 includes an opening (not shown) and is configured to attach to a portion of the braiding machine 36, for example the spindle 38. The distal plate 106 includes an opening 108 and is configured to join to the proximal plate 104 by a plurality of fasteners 105. The proximal plate 104 and the distal plate 106 form a recess or hollowed out portion when joined together. An expanding inner guiding member 110 is located in the recess or hollowed out portion and is affixed to the distal plate 106 by a plurality of fasteners 107.

The expanding inner guiding member 110 is configured to ensure that the axial location of the first and second steering members 28, 30 remains constant along the length of the tubular member 32 during the braiding process. The expanding inner guiding member 110 expands, thereby creating a gap 136, to allow for the passage of the larger diameter pull ring 34 as illustrated in FIG. 10A and then retracts to a neutral or contracted position shown in FIG. 10B that maintains good engagement with the first and second steering members 28, 30 throughout the braiding process.

The expanding inner guiding member 110 has an overall cylindrical shape and includes an upper half 112 (shown in phantom in FIG. 8) and a lower half 114, the upper half 112 and the lower half 114 are semicircular. The expanding inner guiding member 110 includes an outer circumferential groove 116 formed in an outer circumferential edge 118 of the expanding inner guiding member 110. A garter spring 120 is located in the outer circumferential groove and is configured to bias the upper half 112 and lower half 114 together. In other embodiments, springs (not shown) may be oriented radially around the outer circumferential edge 118 that push against an inner surface of the proximal plate 104 or the distal plate 106 to bias the upper half 112 and the lower half 114 together. Additionally, in some embodiments, in lieu of the garter spring 120, the inner guiding member 110 may include magnetic, electromagnetic, or pneumatic devices configured to force the upper half 112 towards the lower half 114 while allowing separation thereof during passage of a larger diameter pull ring 34.

The expanding inner guiding member 110 includes a flared infeed opening 122 and a flared outfeed opening 124. The flared infeed opening 122 and the flared outfeed opening 124 are connected by a constant diameter lumen 126. The flared infeed opening 122, the flared outfeed opening 124, and the constant diameter lumen 126 include a first channel 128 (best viewed in FIGS. 10A and 10B) configured to receive the first steering member 28 and a second channel 130 configured to receive the second steering member 130.

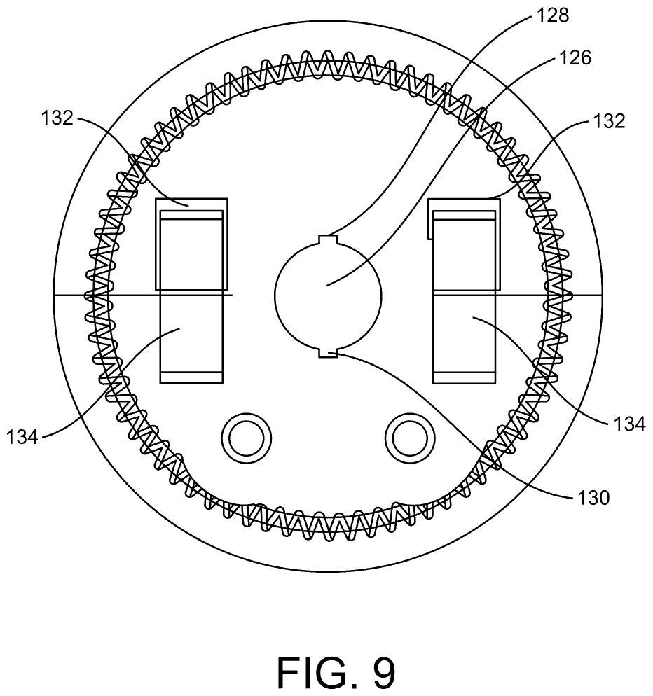

FIG. 9 is a cross-sectional view of the inner guiding member 110 of the nose cone 40 of FIG. 6. As shown in FIG. 9, the expanding inner guiding member 110 includes at least one cylindrical recess 132. The at least one cylindrical recess 132 is formed in both the upper half 112 and the lower half 114. The at least one cylindrical recess 132 is configured to receive at least one cylindrical guide 134. The combination of the cylindrical guide 134 in the cylindrical recess 132 allows for expansion of the inner guiding member 110 along a plane passing through the guides 134 and ensures that the upper half 112 remains aligned with the lower half 114.

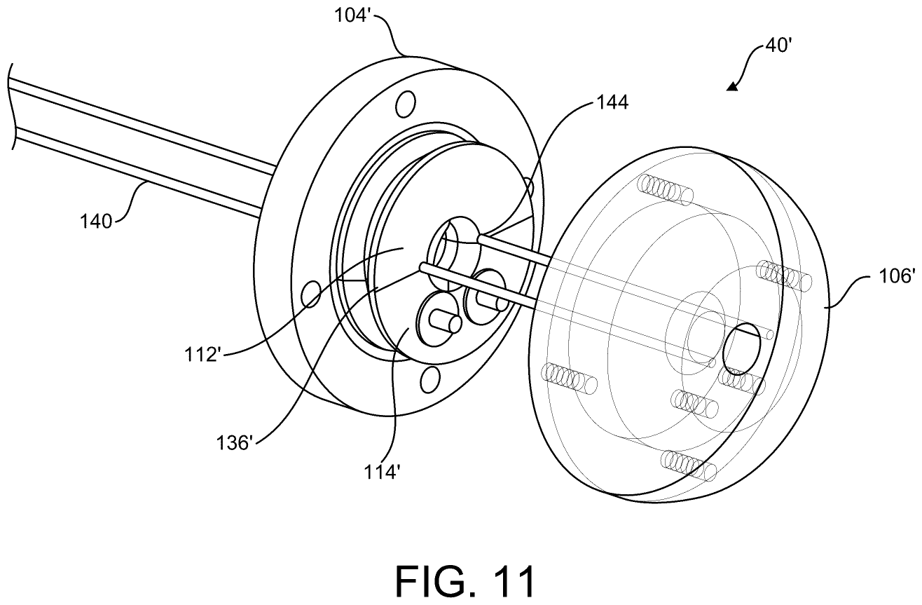

FIG. 11 is a perspective view of an alternative nose cone 40′ for use with the system of FIG. 1, in accordance with embodiments of the disclosure. The nose cone 40′ is similar to the nose cone 40 of FIG. 8 except for the addition of one or more hypotubes 140. The one or more hypotubes 140 extend proximally from the distal plate 106′ and are configured to guide one or more steering member, tube, or sheath associated with a workpiece 26 through a braiding process. The one or more hypotubes 140 are attached to the distal plate 106′ and pass through one or more openings 142 of the expanding inner guiding member 110′ and through one or more openings (not shown) of the proximal plate 104′ The one or more openings 142 of the expanding inner guiding member 110′ are formed through a portion of both the upper half 112′ and the lower half 114′, and are aligned with the gap 136′.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A system for applying a braid to a tubular member, the system comprising:
an infeed clamp configured to prevent rotation of a workpiece, a first steering member, and a second steering member;
an infeed guide configured to maintain the first steering member and the second steering member in a desired axial location along the workpiece; and
a nose cone having a proximal plate, a distal plate, and a radially expanding inner guiding member; wherein the proximal plate and the distal plate form a recess when joined together, and the radially expanding inner guiding member is located in the recess.

2. The system of claim 1, wherein the infeed clamp includes a first steering member retainer, a second steering member retainer, and a workpiece retainer.

3. The system of claim 1, wherein the infeed clamp includes at least one tensioner to apply tension to the first steering member or the second steering member.

4. The system of claim 2, wherein the infeed clamp includes a cover, the cover being biased to an open position allowing for loading of the first steering member into the first steering member retainer, the second steering member into the second steering member retainer, and the workpiece into the workpiece retainer.

5. The system of claim 4, wherein the cover includes a closed position for securing the first steering member in the first steering member retainer, the second steering member in the second steering member retainer, and the workpiece in the workpiece retainer.

6. The system of claim 5, wherein the infeed clamp includes a closure configured to secure the cover in the closed position.

7. The system of claim 6, wherein the infeed guide includes a stationary releasing element configured to initiate release of the closure, allowing the cover to assume the open position.

8. The system of claim 7, wherein the stationary releasing element is spaced from a side of the infeed guide and includes a ramped surface configured for contacting the closure.

9. The system of claim 1, wherein the infeed guide includes an infeed surface and an outfeed surface, and a funnel shaped opening having a first channel configured to receive the first steering member and a second channel configured to receive the second steering member, the funnel shaped opening tapering from the infeed surface to the outfeed surface.

10. The system of claim 1, wherein the expanding inner guiding member includes an upper half and a lower half, the upper half and the lower half being semicircular.

11. The system of claim 10, wherein the expanding inner guiding member includes an outer circumferential groove, and a garter spring located in the outer circumferential groove.

12. The system of claim 10, wherein the expanding inner guiding member includes at least one cylindrical recess, and at least one cylindrical guide located in the at least one cylindrical recess to align the upper half with the lower half.

13. The system of claim 1, wherein the expanding inner guiding member includes a flared infeed opening and a flared outfeed opening, the flared infeed opening and the flared outfeed opening being joined by a constant diameter lumen.

14. The system of claim 13, wherein the flared infeed opening, the flared outfeed opening, and the constant diameter lumen include a first channel configured to receive the first steering member and a second channel configured to receive the second steering member.

15. An infeed clamp for use with a system for applying a braid to a tubular member, the infeed clamp comprising:
a first steering member retainer, a second steering member retainer, and a workpiece retainer; and
a cover having an open position and a closed position, the cover being biased to the open position for allowing loading of the first steering member into the first steering member retainer, the second steering member into the second steering member retainer, and the workpiece into the workpiece retainer; and the closed position configured for securing the first steering member in the first steering member retainer, the second steering member in the second steering member retainer, and the workpiece in the workpiece retainer.

16. The clamp of claim 15, further comprising at least one tensioner to apply tension to the first steering member or the second steering member.

17. The claim of claim 15, further comprising a closure configured to secure the cover in the closed position.

18. A nose cone for use with a system for applying a braid to a tubular member, the nose cone comprising:
a proximal plate configured to attach to a portion of the system for applying a braid to a tubular member;
a distal plate configured to join to the proximal plate; and
a radially expanding inner guiding member; wherein the proximal plate and the distal plate form a recess when joined together, and the radially expanding inner guiding member is located in the recess.

19. The nose cone of claim 18, wherein the expanding inner guiding member includes an upper half movable from a lower half; an outer circumferential groove located in the upper half and the lower half; and a garter spring located in the outer circumferential groove configured to bias the upper half and the lower half together.

20. The nose cone of claim 18, wherein the expanding inner guiding member includes a flared infeed opening and a flared outfeed opening, the flared infeed opening and the flared outfeed opening being joined by a constant diameter lumen; the flared infeed opening, the flared outfeed opening, and the constant diameter lumen include a first channel configured to receive a first steering member and a second channel configured to receive a second steering member.
